(19) United States
(12) Patent Application Publication
(10) Pub. No.: US 2025/0099281 A1
(43) Pub. Date: Mar. 27, 2025

(54) DROP FOOT SOCK APPARATUS

(71) Applicant: THORWEAR INC., San Diego, CA (US)

(72) Inventors: Arni Thor, Carlsbad, CA (US); Pei Hsuan Li, Carlsbad, CA (US)

(21) Appl. No.: 18/826,177

(22) Filed: Sep. 6, 2024

Related U.S. Application Data
(60) Provisional application No. 63/536,806, filed on Sep. 6, 2023.

(51) Int. Cl. A61F 5/01

(52) U.S. Cl. CPC A61F 5/0113

(57) ABSTRACT

Drop foot sock apparatus and methods are disclosed. In one embodiment, the drop foot sock apparatus includes a sock that is configured to fit over a calf muscle on a leg of a user; and a lifting cable system. The lifting cable system includes a calf strap; a tensioning structure disposed on an upper region of the sock, the tensioning structure having a rotary tensioning mechanism disposed thereon; an achilles anchor that is configured to be positioned below the tensioning structure; a dorsal anchor that is positioned anterior to the achilles anchor; a pair of foot anchors being positioned anterior to the dorsal anchor; a pair of calf anchors; and a cable that is attached with the rotary tensioning mechanism, the cable being routed through the achilles anchor, the dorsal anchor, the pair of calf anchors and secured to the pair of calf anchors.

### CROSS-REFERENCE TO RELATED APPLICATIONS

This application claims the benefit of priority to U.S. Provisional Pat. App. Ser. No. 63/536,806 filed Sep. 6, 2023, and entitled “Drop Foot Sock Apparatus and Methods”, the contents of which being incorporated herein by reference in its entirety.

### FIELD OF THE DISCLOSURE

The present disclosure relates generally to the field of the correction of disorders of the limbs or spine by use of braces and other devices to correct alignment or provide support and in one exemplary aspect, to a drop foot sock apparatus and methods for manufacturing and using the same.

### DESCRIPTION OF RELATED ART

Drop foot is a common medical condition that has its source in various different pathological conditions. The condition may be caused by trauma in which the peroneal nerve that innervates the peroneal muscles becomes damaged. Drop foot may also be present following a stroke, or it may be congenital. Many orthotic treatments exist for the treatment of drop foot including, for example: rigid ankle foot orthosis (AFOs); semi-rigid AFOs; soft AFOs (such as “Foot Up”-type devices or a soft ankle brace with straps); and functional electrical stimulation systems.

The primary goal for each of these solutions is in the prevention of plantar flexion of the foot during swing phase, as well as reducing foot slap during heel strike. Considerations that these devices try to take into account are one or more of: (1) improved overall stability of the ankle; (2) the devices ease of donning and doffing (i.e., easy to put on or take off); (3) the devices are comfortable to wear; (4) the devices are aesthetically pleasing in appearance; and/or (5) the devices are easy to wear with shoes. Unfortunately while these devices try to maximize one or more of the benefits listed above, most fail at achieving all of these items, or are otherwise sub-optimal in one or more of these areas. For example, typical complaints by patients with a drop foot condition that are mentioned when utilizing these prior orthopedic devices are: (1) the device is uncomfortable to wear; (2) the device is difficult to integrate into a standard shoe; (3) the device is too big and bulky; (4) the device typically needs to be worn with a specially designed shoe, which isn't always appropriate in all circumstances (e.g., when wearing the device at home); and (5) the device does not provide the proper amount of immobilization (i.e., too much or too little support) for everyday usage.

In recent years, there have been two (2) main types of devices which have particularly tried to tackle these five (5) common complaints; however, these devices have not managed to solve these problems, or other problems have arisen as a result of their designs. One such type of device is a functional electrical stimulation device. While functional electrical stimulation devices are promoted as providing stability only when you need it; being low profile and generally easy to hide; are generally easy to put on and take off; and can be worn with or without shoes, these types of devices aren't without their drawbacks. For example, often times these functional electrical stimulation devices can be uncomfortable to wear; do not always work; and are relatively expensive. Another type of device are so-called “Foot Up” types of devices, that typically include a soft ankle strap that connects to a shoe or a foot strap. These types of devices are typically low profile and easy to hide; are easy to put on and take off; and are generally comfortable. However, these types of devices are not very aesthetically pleasing; offer poor support (e.g., the device slips down on a user's ankle and otherwise loses its ability to provide a supporting function); and preferably need to be worn with shoes.

Accordingly, despite the wide variety of the foregoing solutions, there remains a salient need for an orthotic device that: provides adequate support for everyday use; is comfortable to wear; is inexpensive; is easy to put on and take off; can be worn with and without shoes; and fits well with existing clothing.

### SUMMARY

The present disclosure satisfies the foregoing needs by providing, inter alia, an ankle foot orthosis apparatus for addressing each of the foregoing desirable traits as well as methods of their manufacture and methods of their use.

In one aspect of the present disclosure, an orthosis is disclosed. In one embodiment, the orthosis includes an ankle foot orthosis that includes a sock that is configured to fit over a calf muscle on a leg of a user; and a lifting cable system that is at least partially integrated within the sock. The lifting cable system includes a calf strap that is configured to be positioned above the calf muscle of the user when disposed on the leg of the user; a tensioning structure disposed on an upper region of the sock, the tensioning structure comprising a rotary tensioning mechanism disposed thereon; an achilles anchor that is configured to be positioned below the tensioning structure on posterior portion of the sock; a dorsal anchor that is configured to be positioned anterior to the achilles anchor; a pair of foot anchors being positioned anterior to the dorsal anchor; a pair of calf anchors that are positioned on an upper posterior portion of the sock; and a cable that is attached with the rotary tensioning mechanism, the cable being routed through the achilles anchor, the dorsal anchor, the pair of calf anchors and secured to the pair of calf anchors.

In one variant, the sock includes a first outer layer of sock and a second inner layer of sock, wherein the achilles anchor, the dorsal anchor, and the pair of foot anchors are positioned between the first outer layer of sock and the second inner layer of sock.

In another variant, the AFO includes a toe box positioned anterior to the dorsal anchor on the sock, the toe box being configured to receive toes of the user while wearing the AFO.

In yet another variant, the toe box possesses more elasticity in a circumferential direction around the sock as compared with elasticity in a longitudinal direction, the longitudinal direction generally running from a toe region of the sock toward the dorsal anchor, the circumferential direction being orthogonal with the longitudinal direction.

In yet another variant, the pair of foot anchors wrap around a foot region of the sock such that portions of the pair of foot anchors reside on both a top region of the sock and a bottom region of the sock.

In yet another variant, the pair of foot anchors are disposed between the first outer layer of sock and the toe box.

In yet another variant, the calf strap includes a first end and a second end, wherein the first end and the second end of the calf strap are each configured to be received within respective slots located on the tensioning structure.

In yet another variant, the first end of the calf strap is configured to be attached to a medial side of the sock, and the second end of the calf strap is configured to be attached to a lateral side of the sock.

In yet another variant, the first end of the calf strap is configured to be attached to the calf strap on the medial side of the sock and the second end of the calf and the second end of the calf strap is configured to be attached to the calf strap on the lateral side of the sock.

In yet another variant, an upper portion of the tensioning structure is attached to the upper region of the sock and a lower portion of the tensioning structure is not secured to the sock.

In yet another variant, the rotary tensioning mechanism is secured to the lower portion of the tensioning structure.

In yet another variant, each of the pair of calf anchors comprises a width and the cable is routed through the width of each of the pair of calf anchors before being secured to itself.

In yet another variant, the achilles anchor includes four cable entry points, with two of the four entry points being located on a medial side of the sock and the other two of the four entry points being located on a lateral side of the sock.

In yet another variant, the dorsal anchor includes four cable entry points, with two of the four entry points being located on the medial side of the sock and the other two of the four entry points being located on the lateral side of the sock.

In yet another variant, each of the pair of foot anchors includes a single cable entry point.

In yet another variant, the cable includes a first cable that is routed on the lateral side of the sock and a second cable that is routed on the medial side of the sock.

In yet another variant, the first cable is routed from the rotary tensioning mechanism to a first cable entry point on the achilles anchor, to a first cable entry point on the dorsal anchor, to the single cable entry point on one of the pair of foot anchors, back towards a second cable entry point on the dorsal anchor to a second cable entry point on the achilles anchor and finally to one of the pair of calf anchors.

In yet another variant, the second cable is routed from the rotary tensioning mechanism to a third cable entry point on the achilles anchor, to a third cable entry point on the dorsal anchor, to the single cable entry point on a second of the pair of foot anchors, back towards a fourth cable entry point on the dorsal anchor to a fourth cable entry point on the achilles anchor and finally to a second of the pair of calf anchors.

In yet another variant, the AFO includes pull tabs that are positioned posterior from the pair of foot anchors, the pull tabs being configured to assist with donning and/or doffing of the AFO.

In yet another variant, a portion of the tensioning structure is disposed between the first outer layer of sock and the second inner layer of sock and another portion of the tensioning structure is not covered by the first outer layer of sock.

In another aspect, methods of manufacturing the aforementioned orthosis are disclosed.

In yet another aspect, methods of utilizing the aforementioned orthosis are also disclosed.

In yet another aspect, a finger loop tab is disclosed.

Other features and advantages of the present disclosure will immediately be recognized by persons of ordinary skill in the art with reference to the attached drawings and detailed description of exemplary implementations as given below.

### BRIEF DESCRIPTION OF DRAWINGS

FIG. 1A is a front lateral perspective view of one exemplary orthosis device, in accordance with the principles of the present disclosure.

FIG. 1B is a front medial perspective view of the exemplary orthosis device of FIG. 1A, in accordance with the principles of the present disclosure.

FIG. 1C is a perspective view of an exemplary multi-cable distribution system for use with, for example, the orthosis shown in FIGS. 1A and 1B, in accordance with the principles of the present disclosure.

FIG. 2 is a perspective view of a forefoot wedge insert for use with, for example, the orthosis shown in FIGS. 1A and 1B, in accordance with the principles of the present disclosure.

FIGS. 3A-3D are various views of a finger loop tab for use with, for example, the orthosis shown in FIGS. 1A and 1B, in accordance with the principles of the present disclosure.

FIG. 4A is a front perspective view of another exemplary orthosis device, in accordance with the principles of the present disclosure.

FIG. 4B is a front perspective view of the exemplary orthosis device of FIG. 4A with the top sock layer removed from view, in accordance with the principles of the present disclosure.

FIG. 4C is a rear perspective view of the exemplary orthosis device of FIG. 4B, in accordance with the principles of the present disclosure.

FIG. 5 is a perspective view of a dosing indicator system for use with the orthosis devices described herein, in accordance with the principles of the present disclosure.

FIG. 6 is a perspective view of an exemplary Y-strapping configuration for use with the orthosis devices described herein, in accordance with the principles of the present disclosure.

### DETAILED DESCRIPTION

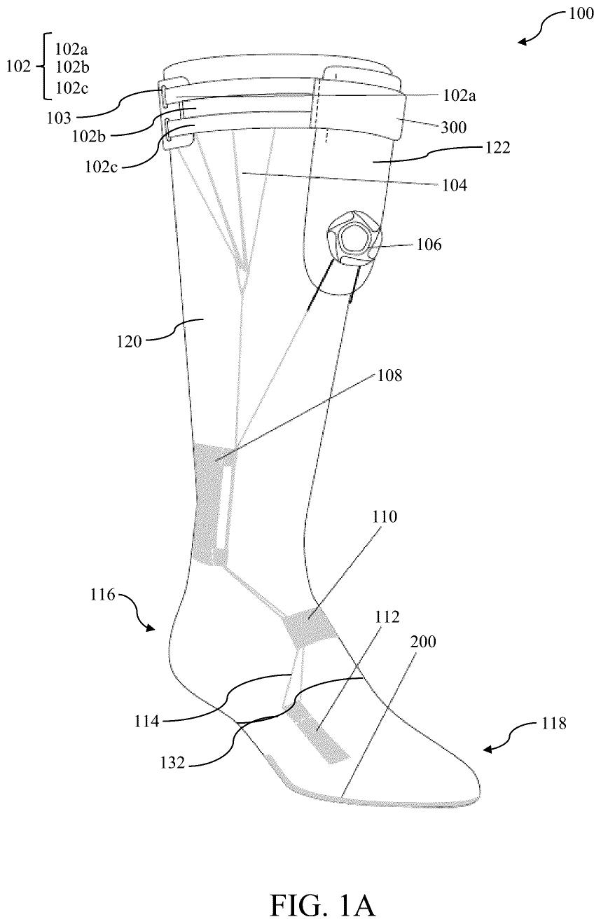

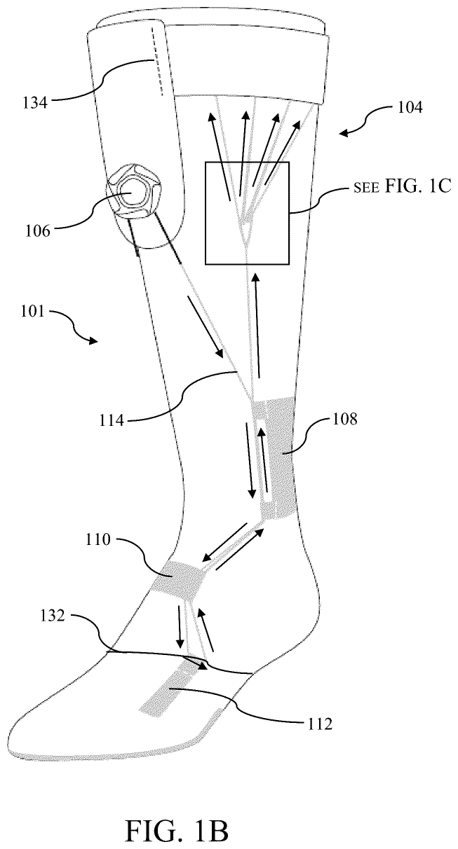

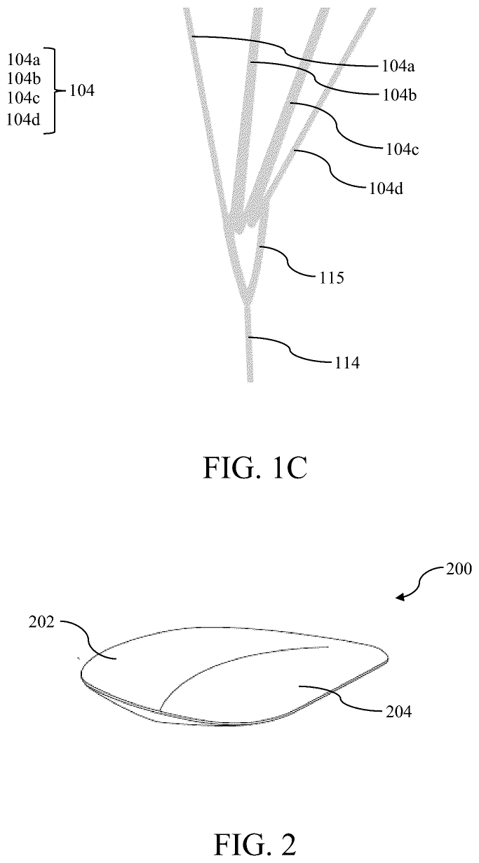

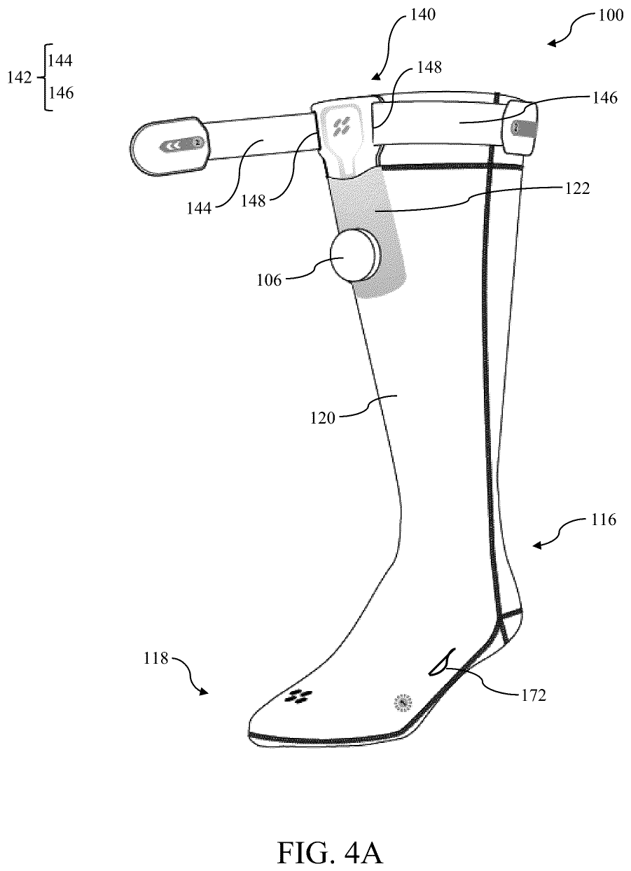

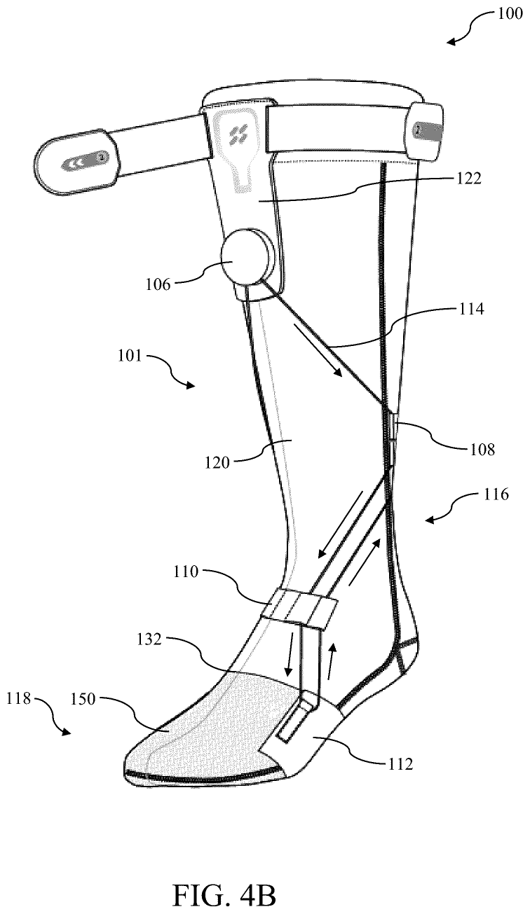

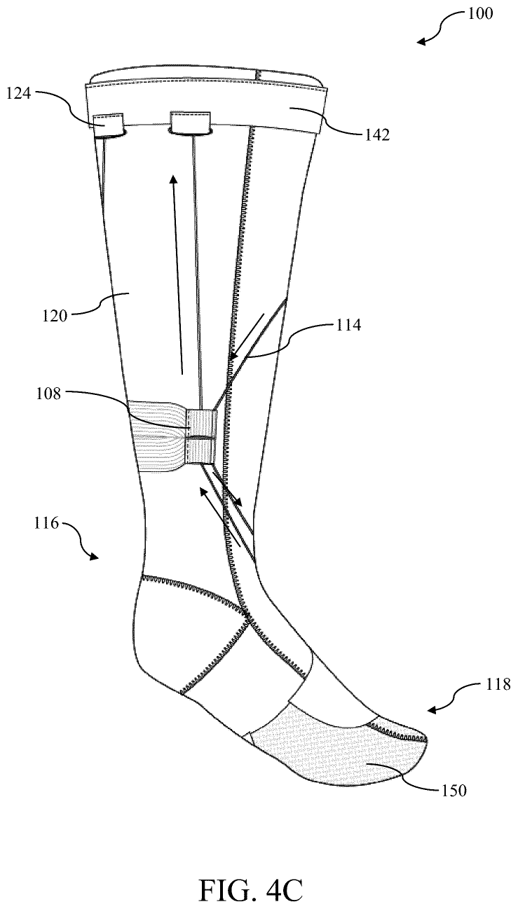

Implementations of the present technology will now be described in detail with reference to the drawings, which are provided as illustrative examples to enable those skilled in the art to practice the technology. Notably, the figures and examples below are not meant to limit the scope of the present disclosure to any single implementation or implementations, but other implementations are possible by way of interchange of, substitution of, or combination with some or all of the described or illustrated elements. For example, many of the features shown and described with respect to the orthosis device of FIGS. 4A-4C may be utilized, where appropriate, with the features shown and described with respect to the features shown in FIGS. 1A-3D, 5, and 6 and vice versa. Wherever convenient, the same reference numbers will be used throughout the drawings to refer to same or like parts.

Moreover, while embodiments described herein are primarily discussed in the context of ankle foot orthoses (“AFOs”) for the treatment of drop foot, it will be recognized by those of ordinary skill that the present disclosure is not so limited. In fact, the principles of the present disclosure described herein may be readily applied to other parts of the anatomy of a human, and for treatment of conditions other than drop foot. For example, many common injuries, such as a partial or complete tear of a tendon (e.g., a biceps tendon, a triceps tendon, an Achilles tendon, and the like), may require an individual to rest the injured tendon, whether surgical or non-surgical treatment is required. Accordingly, the principles described herein may be readily adapted for use with other portions of the anatomy. For example, the drop foot sock devices described herein may be readily adapted for use on the arm, elbow, shoulder, knee, or other anatomy of a wearer where movement, whether in extension or flexion, may need to be constrained in order to facilitate recovery from, for example, an injury or other medical condition.

Referring now to FIGS. 1A-1C, an exemplary ankle foot orthosis (“AFO”) 100 is shown and described in detail. In some implementations, orthosis 100 may be integrated into a sock 120 as illustrated. As illustrated, the sock 120 may consist of an over the calf sock 120 (i.e., the sock is configured to reach over a wearer's, toes, ankle and calf muscle). However, in alternative implementations orthosis 100 may be distinct and separate from an underlying sock 120 and may be utilized (or not utilized) in conjunction with a separate and distinct sock 120. For example, some or all of the lifting cable (or strap) system 101 (e.g., anchors 108, 110, 112; support cable(s) (or straps(s)) 104, 114; multi-strap tightening system 102; and rotary tensioning mechanism 106) may be separable from the sock 120 for the purposes of, for example, enabling the sock 120 to be cleaned independent from the compression strap system. In some implementations, the sock 120 may consist of two (or more) layers. For example, the sock 120 may consist of an inner sock 120a and an outer sock 120b. In some implementations, a majority portion of the lifting cable (or strap) system 101 may be integrated between the inner sock 120a and the outer sock 120b.

The ankle foot orthosis 100 may be utilized as an aid device for drop foot patients; however, such a means for utilization is not the only condition for which ankle foot orthosis 100 may be utilized. For example, ankle foot orthosis 100 may be used as a sleeve to control, for example, swelling/edema in a patient. Additionally, or alternatively, ankle foot orthosis 100 may be utilized to serve as a proprioceptive reminder device in some implementations. For example, for a wearer of the ankle foot orthosis 100 who is dealing with an injury to the lower part of their leg, the ankle foot orthosis 100 may be used as an indicator that, for example, the wearer is over-exerting themselves during rehabilitation exercises, as but one example. In other words, the pressure applied to the lower part of the leg by the ankle foot orthosis 100 may act as a reminder (e.g., such as an increase in pressure caused by swelling as but one example) to a wearer rehabilitating an injury, thereby promoting increased attention to the injury itself. Additionally, ankle foot orthosis 100 may help alleviate fatigue as; for example, the support cable(s) (or strap(s)) 114 are precisely tightened to predetermined (e.g., comfortable) specifications. Ankle foot orthosis (100) may also be utilized to treat plantar fasciitis through the pulling and controlling of the wearer's arch, midfoot and hindfoot, as well as by stretching the big toe of the wearer. These and other applications for the ankle foot orthosis 100 would be readily apparent to one of ordinary skill given the contents of the present disclosure.

The sock 120 (and/or lifting cable (or strap) system 101) may include features that allow for improved mating between the sock 120 and some or all of the lifting cable (or strap) system 101 in implementations in which the sock 120 is separable from some or all of the compression cable (or strap) system 101. For example, the sock 120 and/or lifting cable (or strap) system 101 (or portions thereof) may include hook and loop fasteners (e.g., Velcro®) or other suitable means to removably couple the sock 120 with some or all of the lifting cable (or strap) system 101. The sock 120 may include integrated rubber beads to prevent, inter alia, movement of the sock 120, with respect to the user's leg and/or the lifting cable (or strap) system 101, when a wearer of the sock is in motion. These integrated rubber beads may be integrated within the sock 120 (i.e., for contact against a wearer's skin) and/or may be integrated on an external portion of the sock 120 (e.g., for contact with one or more cables (or straps) 114 of the lifting cable (or strap) system) 101. In some implementations, these integrated rubber beads may be integrated within the lifting cable (or strap) system 101 itself in addition to, or alternatively from, integrated rubber beads on the sock 120 itself (whether internal, and/or external, to the sock 120).

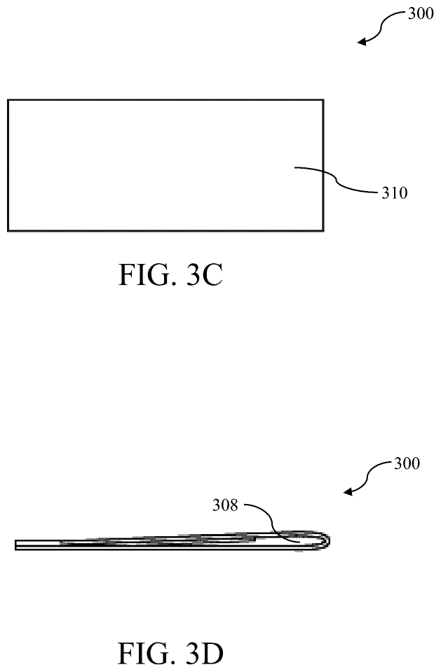

In some implementations, orthosis 100 may include a multi-strap tightening system 102 at an upper portion of the sock 120. As illustrated in FIG. 1A, the multi-strap tightening system 102 includes three (3) distinct straps 102a, 102b, 102c. The center strap 102b may be offset from the outer straps 102a, 102c so that portions of the outer straps 102a, 102c may overlap with the center strap 102b. This overlap prevents the straps 102a, 102b, 102c from separating from one another when tightened, thereby distributing the pressure over the upper calf of the wearer across the total width of the three straps 102a, 102b, 102c. For example, if these straps 102a, 102b, 102c were allowed to separate from one another, individual ones of these straps 102a, 102b, 102c could apply undue pressure to portions of the wearer's upper calf (i.e., akin to placing two or more tourniquets on the upper calf of the wearer) causing discomfort. The straps 102a, 102b, 102c themselves may be manufactured from a suitable webbing material. The straps 102a, 102b, 102c may be connected with the sock 120 and/or the tensioning structure 122, where they travel to distinct slots 103 located on another portion of the multi-strap tightening system 102. This other portion of the multi-strap tightening system 102 with the slots 103 may be manufactured from a thermoplastic polyurethane (TPU), although other suitable materials may be utilized in alternative variants. The straps 102a, 102b, 102c may be threaded through respective slots 103, where they may be connected at the opposite end with the finger loop tab 300 (see also discussion of FIGS. 3A-3D). The finger loop tab 300 enables a user to tighten (or loosen) the straps 102a, 102b, 102c efficiently and easily. For example, the finger loop tab 300 may connect with the tensioning structure via use of, for example, a hook and loop fastener mechanism, buttons, hooks and/or other types of fastening mechanisms. While three (3) distinct straps 102a, 102b, 102c are illustrated in FIG. 1A, it would be appreciated that more (e.g., four (4) or more) (or fewer (e.g., two (2) or less) distinct straps may be implemented in alternative variants.

In some implementations, the tensioning structure 122 may be manufactured from a textile material for comfort. In some implementations, the tensioning structure 122 may have relatively low stretch in the horizontal (or circumferential) direction (i.e., in the direction of the straps 102a, 102b, 102c or circumferentially around the wearer's leg). The low stretch enables the multi-strap tightening system 102 to remain at a desired level of tension when the finger loop tab 300 is attached to the tensioning structure 122. The tensioning structure 122 may also include foam padding in some variants to improve upon comfort for the wearer of the ankle foot orthosis 100. While the use of a textile material for the tensioning structure 122 is exemplary, it would be readily appreciated that the tensioning structure may be made from other materials such as, for example, polymer materials, whether woven or not. The tensioning structure 122 may also include a rotary tensioning mechanism 106 (such as, for example, a BOA® tensioning system). The rotary tensioning mechanism 106 may be utilized to tighten (or loosen) the cable (or strap) 114 as well as, for example, the multi-force distribution system 104. In some implementations, the tensioning structure 122 may be secured only at a top portion of the tensioning structure via sewing, adhesives and the like. See, for example, the stitch line 134 shown in FIG. 1B. Such an arrangement may be advantageous as the rotary tensioning mechanism 106 is allowed to “float” without being secured directly to the sock 120. Accordingly, the sock 120 will not bunch up when tension is applied to the rotary tensioning mechanism 106.

While the use of an exemplary rotary tensioning mechanism 106 is illustrated, it would be readily apparent to one of ordinary skill given the contents of the present disclosure that this mechanism 106 may be obviated in favor of alternatives. For example, the rotary tensioning mechanism 106 may be obviated in favor of a support structure having the cable (or strap) 114 routed therethrough. The support structure may also include a hook and loop fastener disposed thereon, which when routed around, for example, a D-ring positioned on the tensioning structure 122, enables the cable (or strap) 114 to be tightened and secured to the tensioning structure 122. These and other mechanisms for the tightening (or loosening) of the cable (or strap) 114 would be readily apparent to one of ordinary skill given the contents of the present disclosure.

Referring now to FIGS. 1A-1C, components of the lifting cable (or strap) system 101 are shown and described in detail. The lifting cable (or strap) system 101 includes a cable (or strap) 114 as well as a multi-force distribution system 104 that is made up of multiple cables (or straps). As shown in FIGS. 1A and 1B, the multi-force distribution system 104 includes eight (8) distinct cables (or straps), four (4) disposed on the medial side of the sock 120 and four (4) disposed on the lateral side of the sock 120, which distributes the force of the tensioned cable (or strap) 114 around the periphery of the wearer's upper calf muscle. While eight (8) distinct cables (or straps) are illustrated in FIGS. 1A and 1B, the total number cables (or straps) may be more (e.g., ten (10) or more) or less (e.g., six (6) or less) than the illustrated eight (8) distinct cables (or straps).

As shown in FIG. 1C, one-side of the multi-force distribution system 104 is shown which includes the cable (or strap) 114 being connected with the distribution cables (or straps) 104a, 104b, 104c, 104d via a cable (or strap) to distribution cables (or straps) interface 115. As shown, the interface 115 comprises a loop; however, in alternative variants, each of the distribution cables (or straps) 104a, 104b, 104c, 104d may be, for example, spliced with the cable (or strap) 114 so that the interface 115 is not a loop in some implementations. In some implementations, a non-elastic fabric may be attached (e.g., sewn) into an upper portion of the sock 120. There may be no (or a limited amount) of elasticity in the vertical direction (e.g., in the direction stretching vertically from, for example, the calf down towards the ankle), but more elasticity (as compared with the vertical direction) in the horizontal direction (e.g., circumferentially around the user's calf muscle) so that, for example, it does not impact the stretch around the calf of the wearer. These cables (or strap(s)) 104a, 104b, 104c, 104d may anchored to the sock 120 using, for example, grommets or other holes in the sock 120 fabric. In some implementations, these distinct cables (or strap(s)) 104a, 104b, 104c, 104d may be obviated in favor of a single piece of fabric that is triangular in shape (e.g., wider at the upper portion of the sock and narrower as you travel down toward the ankle). In such an implementation, this single piece of fabric may be disposed around the belly of the calf and sewn into the upper portion of the sock 120.

As shown in FIGS. 1A and 1B, the cable (or strap) 114 is routed through a plurality of anchor structures 108, 110, 112, namely an Achilles anchor 108, a dorsal anchor 110, and to one or more different foot anchor(s) 112. These anchor structures 108, 110, 112 may be manufactured from a same (or similar) underlying material, or may alternatively be manufactured from disparate materials. These materials may include, for example, fabrics, rubber-like materials, plastics, resins and/or synthetic materials. Additionally, these anchor structures 108, 110, 112 may be integrated within the sock 120 (i.e., between two or more layers of the sock 120) as shown in FIGS. 1A and 1B. However, in some implementations, these anchor structures 108, 110, 112 (or portions thereof) may be integrated external to the sock 120 and/or integrated internal to all layer(s) of the sock 120.

The Achilles anchor 108 may be integrated within the sock 120 at a position above the ankle region 116 of the sock 120. The Achilles anchor 108 may also wrap around the posterior of the sock 120 such that the Achilles anchor 108 is present on both the medial and the lateral sides of the sock 120. In some implementations, the Achilles anchor 108 protrudes farther anteriorly on the sock 120 on the medial side of the sock 120 as compared with the lateral side of the sock 120. Such an implementation may be advantageous as the Achilles anchor 108 as well as the cable (or strap) 114 running therethrough avoids bony structures on the anatomy of the wearer. However, in some implementations, the Achilles anchor 108 may be identical on both sides of the ankle so that one may use the same sock 120 on both the left and right legs.

In some implementations, it may be desirable to have socks 120 with an adjustable Achilles anchor 108 (or an adjustable length Achilles anchor 108), or to have differing socks 120 with different height adjustments for the Achilles anchor 108 (or different length Achilles anchors 108). For example, with patients that have ankles with smaller circumference, the cables (or straps) 114 may be allowed to pull away from the foot a bit more so being able to shorten the posterior strap will bring the cables (or straps) 114 back down (e.g., closer to the anatomy of the leg). Conversely, for patients with larger ankle structures, the cables (or straps) 114 may tend to get pushed into the foot. In such an instance, lengthening that cable (or strap) 114 may be beneficial. In either instance, it may be beneficial for the cable (or strap) 114 to rest anterior to both malleolus as this may act as the leverage arm created to pull the ankle into dorsiflexion. In other words, If the cable (or strap) 114 went straight through the center of the wearer's malleolus you may not get sufficient dorsiflexion lift, depending upon the condition of the patient. Conversely, if the cables (or straps) 114 went posterior to the malleolus, you may pull the foot into plantar flexion, which may be undesirable dependent upon the condition of the patient.

The Achilles anchor 108 may also include four (4) interface points for the cable (or strap) 114 with two (2) of these positioned on the lateral side of the sock 120 and the other two (2) being positioned on the medial side of the sock 120. These interface points may be constructed to provide a low-friction surface for the cable (or strap) 114 so as to enable the cable (or strap) 114 to slide easily therethrough. As illustrated, the Achilles anchor 108 is integrated between two or more layers of the sock 120 and prevents the cable (or strap) 114 from “lifting”, which could cause the cable (or strap) 114 to lift through the sock 120 and be uncomfortable and/or aesthetically unappealing.

The dorsal anchor 110 may be positioned anterior of the Achilles anchor 108 of the sock 120 towards the toes of the sock 120 (i.e., in the midfoot region on the top of the foot of the wearer of the ankle foot orthosis 100). The dorsal anchor 110 may also be positioned about the mid-line of the sock 120 such that the interface regions of the dorsal anchor 110 (i.e., the portions of the dorsal anchor which interface with the cable (or strap) 114) are positioned internal to the Achilles anchor 108. The dorsal anchor 110 may also include two (2) or more interface points for the cable (or strap) 114 with at least one of these interface points being positioned on the lateral side of the sock 120 and the at least other one being positioned on the medial side of the sock 120. These interface points may be constructed to provide a low-friction surface for the cable (or strap) 114 so as to enable the cable (or strap) 114 to slide easily therethrough. As illustrated, the dorsal anchor 110 is integrated between two or more layers of the sock 120 and prevents the cable (or strap) 114 from “lifting” and/or “spreading”, which could cause the cable (or strap) 114 to lift through the sock 120 and be uncomfortable and/or aesthetically unappealing.

The ankle foot orthosis 100 may also include two (2) foot anchors 112 that are positioned anterior of the dorsal anchor 110 of the sock 120 (i.e., towards the toe region of the sock 120). One of the two (2) foot anchors 112 is positioned on the lateral side of the sock 120, while the other of the two (2) foot anchors 112 is positioned on the medial side of the sock. These foot anchors 112 may also be positioned about the mid-line of the sock 120 such that the interface regions for each of the foot anchors 112 are positioned external to the dorsal anchor 110 (i.e., one foot anchor 112 being positioned medially from the dorsal anchor 110 and the other foot anchor 112 being positioned laterally from the dorsal anchor 110). Each foot anchor 112 may also include one (or more) interface points for the cable (or strap) 114 with at least one of these interface points being positioned on the posterior side of the foot anchor 112. These interface points may be constructed to provide a low-friction surface for the cable (or strap) 114 so as to enable the cable (or strap) 114 to slide easily therethrough. As illustrated, the foot anchors 112 are integrated between two or more layers of the sock 120 and are the lifting points for lifting the forefoot of the wearer when the cable (or strap) 114 is tightened.

Additionally, FIG. 1B illustrates how the cable (or strap) 114 is routed amongst the anchors as indicated by the arrows shown in FIG. 1B. For example, the cable (or strap) 114 may be routed from the rotary tensioning mechanism 106 to the Achilles anchor 108. From the Achilles anchor 108, the cable (or strap) 114 is routed to the dorsal anchor 110, where it passes through the interface regions of the dorsal anchor 110 to the foot anchor 112. The cable (or strap) 114 is then routed through the interface region of the foot anchor 112 back towards the dorsal anchor 110, and then through the interface region of the dorsal anchor 110 towards the Achilles anchor 108. After being routed through the interface region of the Achilles anchor 108, the cable (or strap) 114 is then routed upward towards the multi-force distribution system 104 as discussed supra. The cable (or strap) 114 may also be routed similarly on the opposing side of the sock 120 that is obscured from view in FIG. 1B.

Accordingly, when the cable (or strap) 114 is tightened, a force is applied to the foot anchor(s) 112 which raises the toes of the wearer of the sock 120. Simultaneously, the multi-force distribution system 104 applies a plurality of distinct forces around the circumference of the top of the sock 120, which assists in maintaining the top of the sock 120 above the calf muscle of the wearer. In some implementations, the interface regions of the Achilles anchor 108 and the dorsal anchor 110 may include two (2) (or more) distinct channels. For example, one channel may be utilized to support the cable (or strap) 114 traveling in one direction with respect to the Achilles anchor 108 and dorsal anchor 110, while the another distinct channel may be utilized to support the cable (or strap) 114 traveling in the opposite direction with respect to the Achilles anchor 108 and dorsal anchor 110. In some implementations, the Achilles anchor 108 and dorsal anchor 110 may include a single channel for the routing of the cable (or strap) 114 to/from the Achilles anchor 108 and the dorsal anchor 110. In an alternative implementation, one of either the Achilles anchor 108 or the dorsal anchor 110 may include two (2) distinct channels, while the other one of either the Achilles anchor 108 or the dorsal anchor 110 may include one distinct channel. As illustrated in FIG. 1B, the cable (or strap) 114 enters the foot anchor 112 from the mid-line side of the foot anchor 112 and exits from the opposing side; however, in some implementations this directionality may be reversed.

The underlying sock 120 may, in some implementations, include low stretch in the horizontal direction at least in the region of the ankle and at the top of the calf (i.e., in the region of the multi-strap tightening system 102). The underlying sock 120 may include a higher amount of stretch in the horizontal direction in one or more other regions to enable orthosis 100 to be put on, and taken off, easily, while also providing for improved comfort. Orthosis 100 may also include low stretch in the vertical direction to ensure proper stability of the cable (or strap) 114. The sock 120 may be manufactured from two or layers of material so that the lifting cable (or strap) system 101 (e.g., anchors 108, 110, 112); support cable(s) (or straps(s)) 104, 114 may be integrated between at least two of the layers of material. For example, as illustrated in FIGS. 1A and 1B, those portions of the lifting cable (or strap) system 101 (e.g., anchors 108, 110, 112; support cable(s) (or straps(s)) 104, 114 shown as shaded are integrated within the two or layers of material of the sock 120 with only the portion of the cable (or strap) 114 shown in darker line closer to the rotary tensioning mechanism 106 being disposed outside of the sock 120. At the forefoot 118 of the sock 120, the region from the dividing line 132 towards the toes of the sock 120 may include a material that has little to no stretch in the direction of pulling from the support cable(s) (or straps(s)) 104 and minimal stretch in the perpendicular direction to prevent pull on just the toes of the wearer. Such an implementation may allow for more even lift/pull of the toes and forefoot of the wearer.

In some implementations, the inner layer(s) of the sock 120 (i.e., those layer(s) disposed adjacent to the wearer's skin) may be manufactured from an anti-microbial material to prevent bacterial build up when the orthosis 100 is worn. The two or more layers of material for the sock 120 may be bonded together using, for example, stitching and/or a hot melt adhesive to make the orthosis 100 easier to don or doff. For example, the heel region of the sock 120 may be bonded together using a hot melt adhesive to prevent the sock layers from slipping past one another during donning or doffing of the sock 120. The sock 120 may incorporate nano-fabrics which are textiles that are engineered to provide advantageous material properties such as superhyrdophobicity; odor or moisture elimination, increased elasticity and/or strength as compared with other common textile materials such as cotton. Utilization of nano-fabrics may also provide for a degree of bacterial resistance. The sock 120 may incorporate neoprene materials into regions of the sock 120, such as between the toe region of the sock 120 and the dorsal anchor 110 in order to take advantage of the known properties of neoprene. The opening at the top of the sock 120 may incorporate the aforementioned rubber beads for the purpose of providing slip prevention when the lifting cable (or strap) system 101 (e.g., anchors 108, 110, 112); support cable(s) (or straps(s)) 104, 114 is tightened in order to reduce the possibility of sock slippage. The underside of the sock 120 (i.e., underneath the wearer's feet) may incorporate beads (or grips) made of, for example, silicon to improve traction for wearer of orthosis 100 when not wearing shoes.

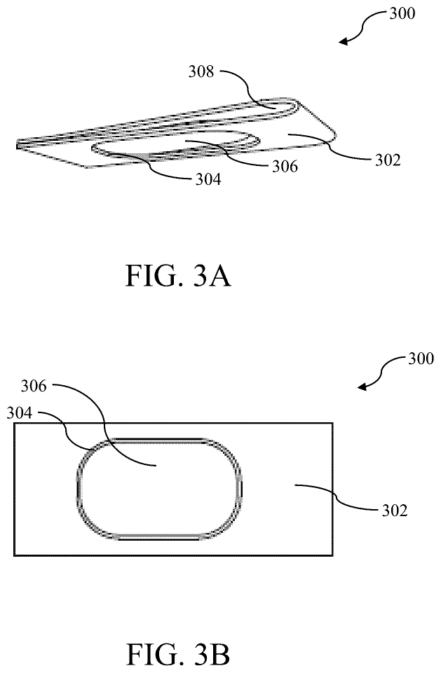

Referring now to FIG. 2, the sock 120 may incorporate a forefoot wedge insert 200 in some implementations. The forefoot wedge insert 200 may be disposed internal to the sock 120, between two or more layers of the sock 120, or may be secured to the outer layer of the sock 120 using, for example, stitching techniques or hot melt adhesives. The forefoot wedge insert 200 may consist of a forefoot cup 204 as well as a toe cup 202. The forefoot wedge insert 200 may assist wearers whose toes may have a tendency to curl via utilization of a rigid or semi-rigid toe cup 202 that positions the wearer's toes upwards. The forefoot wedge insert 200 may be rigid (or somewhat rigid) such that the forefoot wedge insert 200 has the ability to hold the toes of the wearer upward. For example, the forefoot wedge insert 200 may be manufactured from a rigid foam and/or a flexible and/or semi rigid plastic and/or from a rigid plastic in some implementations.

In some implementations, the lifting cable (or strap) system 101 may be integrated onto other wearable support structures other than the ankle foot orthosis 100 explicitly shown. For example, in the context of an arm and elbow orthosis (not shown), retention structures may be placed above the bicep, above and/or below the elbow and at the wrist as but one exemplary example. As but another exemplary example in the context of a leg and knee orthosis, retention structures may be placed at the top of the thigh, above and/or below the knee and below the calf. In sum, various embodiments may be envisioned for use on various portions of the human anatomy with anchor structures (e.g., anchor points) being positioned at various portions of the anatomy which go from either larger diameter to smaller diameter or from smaller diameter to larger diameter. These and other variations would be readily apparent to one of ordinary skill given the contents of the present disclosure.

Additional padding (and/or support) may be provided underneath (or around) anchor structure(s) 108, 110 and/or 112 dependent upon, for example, a particular user's preference for the purpose of providing additional comfort. The underside of the foot may also include padded areas (e.g., via the addition of foam, fabric and/or rubber, etc.) for added comfort. The addition of these padded areas may help prevent the user from feeling excess tension as the anchor structure(s) 108, 110 and/or 112 are placed under tension and may also be configured for a particular user's preferences.

Referring now to FIGS. 3A-3D, an exemplary finger loop tab 300 for use with, for example, the multi-strap tightening system 102, is shown and described in detail. The finger loop tab 300 is manufactured from a material 302 that is folded over on itself to form a gap 308. This material 302 may consist of webbing or other soft pliable materials that enable the gap 308 to be closed when securing the finger loop tab 300 onto the tensioning structure 122 of the ankle foot orthosis 100. The finger loop tab 300 may include an aperture 304 positioned on one side of the finger loop tab 300. In some implementations, a hook and loop fastener 306 may be positioned within the aperture 304. The hook and loop fastener 306 may be configured to interact with hook and loop fastening material located on the tensioning structure 122. The gap 308 within the finger loop tab 300 is to prevent the hook and loop fastener 306 from engaging with or otherwise damaging other articles of clothing such as, for example, the body of the sock 120. However, when the finger loop tab 300 is desired to be attached with the tensioning structure 122, a user may pull the finger loop tab 300 taught over the tensioning structure 122 and then may depress the top surface 310 of the finger loop tab 300 positioned over the aperture 304 and may engage the hook and loop fastener 306 with a similar material located on the tensioning structure 122.

Referring now to FIGS. 4A-4C, yet another ankle foot orthosis 100 is shown and described in detail. As shown in FIG. 4A, the ankle foot orthosis 100 includes a double slot calf support system 140. The double slot calf support system 140 includes a tensioning structure 122 having a rotary tensioning mechanism 106 disposed thereon. The tensioning structure 122/double slot calf support system 140 may also include a pair of slots 148. In some implementations, the tensioning structure 122 may be semi-rigid with a flexible center portion that allows the tensioning structure 122 to wrap around the tibia of the wearer of the ankle foot orthosis 100. The ankle foot orthosis 100 may also include a calf strap 142 that may be attached to the sock 120 in, for example, a posterior portion of the sock 120. The calf strap 142 may include two free ends, namely a medial portion end 144 of the calf strap 142 as well as a lateral portion end 146 of the calf strap 142. The two free ends 144, 146 of the calf strap 142 may be fed through the slots 148 located on the tensioning structure 122 which enables a user to pull on each of the free ends 144, 146 of the calf strap 142 to provide a circumferential tightening of the calf strap 142 around the top of the wearer's calf. The calf strap 142 may also include some elasticity to assist with suspension as well as comfort for the wearer of the ankle foot orthosis 100. The sock 120 may also include one or more pull tabs 172 (e.g., one positioned on the medial side with another positioned on the lateral side) which may assist with donning or doffing of the ankle foot orthosis 100. In some implementations, the tensioning structure 122 may be secured only at a top portion (e.g., in the portion adjacent to the slots 148) of the tensioning structure 122 via sewing, adhesives and the like. See also, for example, the stitch line 134 shown in FIG. 1B. Such an arrangement may be advantageous as the rotary tensioning mechanism 106 is allowed to float without being secured directly to the sock 120 itself, meaning that, the sock 120 will not bunch up when tension is applied to the rotary tensioning mechanism 106.

Referring now to FIG. 4B, a lifting cable (or strap) system 101 may be integrated between two or more layers of a sock 120 as illustrated (the top layer(s) being removed from FIG. 4B). As illustrated, the sock 120 may consist of an over the calf sock 120 (i.e., the sock is configured to reach over a wearer's, toes, ankle and calf muscle). In some implementations, the lifting cable (or strap) system 101 (or portions thereof) may be distinct and separate from an underlying sock 120 and may be utilized (or not utilized) in conjunction with a separate and distinct sock 120. For example, the lifting cable (or strap) system 101 (e.g., anchors 108, 110, 112, 124; support cable(s) (or straps(s)) 114; calf strap 142; and rotary tensioning mechanism 106) or portions thereof may be separable from the sock 120 for the purposes of, for example, enabling the sock 120 to be cleaned independent from the compression strap system 101. In some implementations, the sock 120 may consist of two (or more) layers. For example, the sock 120 may consist of an inner sock layer and an outer sock layer. In some implementations, a majority portion of the lifting cable (or strap) system 101 may be integrated between an inner sock layer and an outer sock layer.

As shown in FIG. 4B, the cable (or strap) 114 is routed through a plurality of anchor structures 108, 110, 112, 124, namely an Achilles anchor 108, a dorsal anchor 110, a foot anchor 112 (e.g., two (2)), and one or more calf anchors 124 (e.g., two (2)). These anchor structures 108, 110, 112, 124 may all be manufactured from the same (or similar) underlying material or may alternatively be manufactured from disparate materials. These materials may include, for example, fabrics (e.g., webbing-like materials with varying gradations of elasticity), rubber-like materials, plastics, resins and/or synthetic materials. Additionally, these anchor structures 108, 110, 112, 124 may be integrated within the sock 120 (i.e., between two or more layers of the sock 120) and attached to one or more layers of the sock through sewing, adhesives, and the like. In some implementations, these anchor structures 108, 110, 112, 124 (or portions thereof) may be integrated external to the sock 120 and/or integrated internal to all layer(s) of the sock 120.

The Achilles anchor 108 may be integrated within the sock 120 at a position above the ankle region 116 of the sock 120. The Achilles anchor 108 may also wrap around the posterior of the sock 120 such that the Achilles anchor 108 is present on both the medial and the lateral sides of the sock 120. In some implementations, the Achilles anchor 108 protrudes farther anteriorly on the sock 120 on the medial side of the sock 120 as compared with the lateral side of the sock 120. Such an implementation may be advantageous as the Achilles anchor 108 as well as the cable (or strap) 114 running therethrough avoids bony structures on the anatomy of the wearer. However, in some implementations, the Achilles anchor 108 may be identical on both sides of the ankle so that one may use the same sock 120 on both the left and right legs.

In some implementations, it may be desirable to have socks 120 with an adjustable Achilles anchor 108 (or an adjustable length Achilles anchor 108), or to have differing socks 120 with different height adjustments for the Achilles anchor 108 (or different length Achilles anchors 108). For example, with patients that have ankles with smaller circumference, the cables (or straps) 114 may be allowed to pull away from the foot a bit more so being able to shorten the posterior strap will bring the cables (or straps) 114 back down (e.g., closer to the anatomy of the leg). Conversely, for patients with larger ankle structures, the cables (or straps) 114 may tend to get pushed into the foot. In such an instance, lengthening that cable (or strap) 114 may be beneficial. In either instance, it may be beneficial for the cable (or strap) 114 to rest anterior to both malleolus as this may act as the leverage arm created to pull the ankle into dorsiflexion. In other words, If the cable (or strap) 114 went straight through the center of the wearer's malleolus you may not get sufficient dorsiflexion lift, depending upon the condition of the patient. Conversely, if the cables (or straps) 114 went posterior to the malleolus, you may pull the foot into plantar flexion, which may be undesirable dependent upon the condition of the patient.

The Achilles anchor 108 may also include four (4) interface points for the cable (or strap) 114 with two (2) of these positioned on the lateral side of the sock 120 and the other two (2) being positioned on the medial side of the sock 120. These interface points may be constructed to provide a low-friction surface for the cable (or strap) 114 to enable the cable (or strap) 114 to slide easily therethrough. As illustrated, the Achilles anchor 108 is integrated between two or more layers of the sock 120 and prevents the cable (or strap) 114 from “lifting”, which could cause the cable (or strap) 114 to lift through the sock 120 and be uncomfortable and/or aesthetically unappealing.

The dorsal anchor 110 may be positioned anterior of the Achilles anchor 108 of the sock 120 towards the toes of the sock 120 (i.e., in the midfoot region on the top of the foot of the wearer of the ankle foot orthosis 100). The dorsal anchor 110 may also be positioned about the mid-line of the sock 120 such that the interface regions of the dorsal anchor 110 (i.e., the portions of the dorsal anchor which interface with the cable (or strap) 114) are positioned internal to the Achilles anchor 108. The dorsal anchor 110 may also include two (2) or more interface points for the cable (or strap) 114 with at least one of these interface points being positioned on the lateral side of the sock 120 and the at least other one being positioned on the medial side of the sock 120. These interface points may be constructed to provide a low-friction surface for the cable (or strap) 114 to enable the cable (or strap) 114 to slide easily therethrough. As illustrated, the dorsal anchor 110 is integrated between two or more layers of the sock 120 and prevents the cable (or strap) 114 from “lifting” and/or “spreading”, which could cause the cable (or strap) 114 to lift through the sock 120 and be uncomfortable and/or aesthetically unappealing.

The ankle foot orthosis 100 may also include two (2) foot anchors 112 that are positioned anterior of the dorsal anchor 110 of the sock 120 (i.e., towards the toe region 118 of the sock 120). One of the two (2) foot anchors 112 is positioned on the lateral side of the sock 120, while the other of the two (2) foot anchors 112 is positioned on the medial side of the sock. These foot anchors 112 may also be positioned about the mid-line of the sock 120 such that the interface regions for each of the foot anchors 112 are positioned external to the dorsal anchor 110 (i.e., one foot anchor 112 being positioned medially from the dorsal anchor 110 and the other foot anchor 112 being positioned laterally from the dorsal anchor 110). Each foot anchor 112 may also include one (or more) interface points for the cable (or strap) 114 with at least one of these interface points being positioned on the posterior side of the foot anchor 112. These interface points may be constructed to provide a low-friction surface for the cable (or strap) 114 to enable the cable (or strap) 114 to slide easily therethrough. As illustrated, the foot anchors 112 are integrated between two or more layers of the sock 120 and are the lifting points for lifting the forefoot of the wearer when the cable (or strap) 114 is tightened. The foot anchors 112 may also be sized so that these foot anchors 112 wrap around the foot to be present on both the top portion of the foot as well as the bottom portion of the foot to assist with the lifting of the foot.

At the forefoot 118 of the sock 120, the region from the dividing line 132 towards the toes of the sock 120 may include a so-called toe box 150, manufactured from a material that has little to no stretch in the direction of pulling from the support cable(s) (or straps(s)) 114 and minimal stretch in the perpendicular direction to prevent pull on just the toes of the wearer. Such an implementation may allow for more even lift/pull of the toes and forefoot of the wearer. As a brief aside, the toe box 150 captures the forefoot of the wearer from the toe region 118 past the metatarsals of the wearer. By minimizing elasticity in the longitudinal region (i.e., towards the dorsal anchor 110, while enabling some degree of elasticity in the circumferential direction around the wearer's foot, the comfort and effectiveness of the ankle foot orthosis 100 may be improved. For example, if the toe box 150 were too “stretchy” in the longitudinal direction, the cable (or strap) 114 would direct most of the force applied to the foot anchors 112 at the toes of the wearer and would not effectively pull the ankle into dorsiflexion making ankle foot orthosis 100 uncomfortable to wear. By minimizing stretch in the longitudinal direction, the force is distributed more evenly across the entire forefoot region so that the “pull” from the cable is focused towards pulling the forefoot into dorsiflexion. The circumferential elasticity however allows the sock to accommodate different bony prominences and allows for wider adjustability in sizing for different user anatomies.

For example, the foot anchors 112 may also be positioned over the top of a mesh-like material that forms the toe box 150 in some implementations as illustrated in FIG. 4B. The mesh-like material 150 may be disposed in the toe region 118 and may have stretch in the circumferential direction around the foot to assist with, for example, donning and doffing of the sock 120, while having little to no elasticity in the longitudinal direction (i.e., from the toe region 118 back towards the dorsal anchor 110). By having little to no elasticity in the longitudinal direction, the mesh-like material may “cradle” the metatarsals of the wearer's foot providing for increased comfort and surface area when the toes of the wearer are lifted via, for example, the tightening of the cable (or strap) 114. As can be seen in FIG. 4C, the mesh-like material 150 also covers portions of the underside of the foot (e.g., the ball of the foot) which assists with the lifting of the wearer's toes.

Additionally, FIGS. 4B and 4C illustrate how the cable (or strap) 114 is routed amongst the anchors as indicated by the arrows shown in FIGS. 4B and 4C. For example, the cable (or strap) 114 may be routed from the rotary tensioning mechanism 106 to the Achilles anchor 108. From the Achilles anchor 108, the cable (or strap) 114 is routed to the dorsal anchor 110, where it passes through the interface regions of the dorsal anchor 110 to the foot anchor 112. The cable (or strap) 114 is then routed through the interface region of the foot anchor 112 back towards the dorsal anchor 110, and then through the interface region of the dorsal anchor 110 towards the Achilles anchor 108. After being routed through the interface region of the Achilles anchor 108, the cable (or strap) 114 is then routed upward (see FIG. 4C) towards a multi-force distribution system 104 as discussed supra, or towards the calf anchors 124 positioned on the posterior portion of the sock 120 (as illustrated in FIG. 4C), near the top portion of the sock 120. The cable (or strap) 114 may also be routed similarly on the opposing side of the sock 120 that is obscured from view in FIGS. 4B and 4C.

Accordingly, when the cable (or strap) 114 is tightened, a force is applied to the foot anchor(s) 112 which raises the toes of the wearer of the sock 120. Simultaneously, calf anchors 124 applies a plurality of distinct forces around the circumference of the top of the sock 120 (towards a posterior portion of the sock), which assists in maintaining the top of the sock 120 above the calf muscle of the wearer. As a brief aside, by having the cable (or strap) 114 run between the rotary tensioning mechanism 106 (located on the front of the wearer's leg) and the two (or more) calf anchors 124 positioned towards the back of the wearer's calf, there is a half reduction in force (or more due to friction), as well as the fact that the forces applied due to tightening of the cable (or strap) 114 are used to compress the sock 120 itself around the user's calf muscle, the sock 120 is resistant to being pulled down off the calf muscle of the wearer. The use of the aforementioned multi-force distribution system (104, FIGS. 1B and 1C) distributes the load around the wearer's calf resulting in an ability to retain the sock 120 above the calf muscle, even when the cable (or straps) 114 are tightened. In some implementations (as shown in FIG. 4C), the cable (or strap) 114 can be routed and secured to a larger piece of fabric (e.g., calf anchor 124) which assists with spreading of the load resultant from the tightened cables (or straps) 114 over a larger area which enables a single cable (or strap) to effectively exert a load over a larger area. The cable (or strap) 114 may also dynamically tighten when the cable (or strap) 114 tightens to create a dynamic tensioning system. Such dynamic tensioning may create an exercise device to help increase blood flow and muscle strengthening of the calf muscle which may often be weakened with some patients.

In some implementations, the interface regions of the Achilles anchor 108 and the dorsal anchor 110 may include two (2) (or more) distinct channels. For example, one channel may be utilized to support the cable (or strap) 114 traveling in one direction with respect to the Achilles anchor 108 and dorsal anchor 110, while the other distinct channel may be utilized to support the cable (or strap) 114 traveling in the opposite direction with respect to the Achilles anchor 108 and dorsal anchor 110. In some implementations, the Achilles anchor 108 and dorsal anchor 110 may include a single channel for the routing of the cable (or strap) 114 to/from the Achilles anchor 108 and the dorsal anchor 110. In an alternative implementation, one of either the Achilles anchor 108 or the dorsal anchor 110 may include two (2) distinct channels, while the other one of either the Achilles anchor 108 or the dorsal anchor 110 may include one distinct channel. As illustrated in FIG. 4B, the cable (or strap) 114 enters the foot anchor 112 from the mid-line side of the foot anchor 112 and exits from the opposing side; however, in some implementations this directionality may be reversed.

The underlying sock 120 may, in some implementations, include low stretch in the horizontal direction at least in the region of the ankle and at the top of the calf (i.e., in the region of the calf strap 142). The underlying sock 120 may include a higher amount of stretch in the horizontal direction in one or more other regions to enable orthosis 100 to be put on, and taken off, easily, while also providing for improved comfort. Orthosis 100 may also include low stretch in the vertical direction to ensure proper stability of the cable (or strap) 114.

In some implementations, the inner layer(s) of the sock 120 (i.e., those layer(s) disposed adjacent to the wearer's skin) may be manufactured from an anti-microbial material to prevent bacterial build up when the orthosis 100 is worn. The two or more layers of material for the sock 120 may be bonded together using, for example, stitching and/or a hot melt adhesive to make the orthosis 100 easier to don or doff. For example, the heel region of the sock 120 may be bonded together using a hot melt adhesive to prevent the sock layers from slipping past one another during donning or doffing of the sock 120. The sock 120 may incorporate nano-fabrics which are textiles that are engineered to provide advantageous material properties such as superhyrdophobicity; odor or moisture elimination, increased elasticity and/or strength as compared with other common textile materials such as cotton. Utilization of nano-fabrics may also provide for a degree of bacterial resistance. The sock 120 may incorporate neoprene materials into regions of the sock 120, such as between the toe region of the sock 120 and the dorsal anchor 110 to take advantage of the known properties of neoprene. The opening at the top of the sock 120 may incorporate the aforementioned rubber beads for the purpose of providing slip prevention when the lifting cable (or strap) system 101 (e.g., anchors 108, 110, 112, 124); support cable(s) (or straps(s)) 104, 114 is tightened in order to reduce the possibility of sock slippage. The underside of the sock 120 (i.e., underneath the wearer's feet) may incorporate beads (or grips) made of, for example, silicon to improve traction for wearer of orthosis 100 when not wearing shoes.

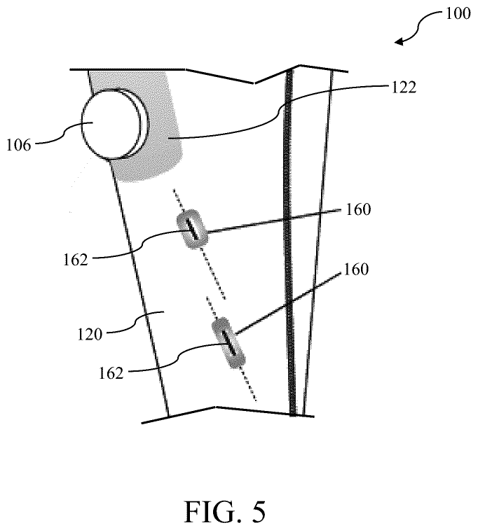

Referring now to FIG. 5, in some implementations, ankle foot orthosis 100 may also include a dosing indicator system. The dosing indicator system enables the wearer to tighten the support cable (or strap) 114 to a prescribed level of tension by, for example, a clinician. In some implementations, the dosing indicator system includes one or more dosing indicators 160 present on the sock 120 as well as one or more cable indicators 162 present on the support cable (or strap) 114. However, in addition (or alternatively) from the visual indicators shown in FIG. 5, the dosing indicator system could include auditory and/or haptic feedback in some implementations. One advantage for the dosing indicator system is that the wearer of the ankle foot orthosis 100 would have an indicator that clearly shows how much tension should be applied to the support cable (or strap) 114 for the ankle foot orthosis 100. As illustrated in FIG. 5, the dosing indicator system may include a dosing indicator 160 on the sock 120 which is an aperture present within the sock 120 that allows the cable indicator 162 to be seen when an appropriate level of tensioning is achieved. In implementations that include multiple dosing indicators 160 and one or more cable indicators 162, different levels of tensioning can be precisely labeled for use in different situations (e.g., in the morning, afternoon and/or evening, during different levels of intensity of movement for the wearer and/or other appropriate activity conditions). In some implementations, a tension meter could be included on ankle foot orthosis 100 to indicate the amount of tension applied to the support cable (or strap) 114 in, for example, Newtons that would then translate to Newton-meters (Nm) of moment applied to the ankle of the wearer.

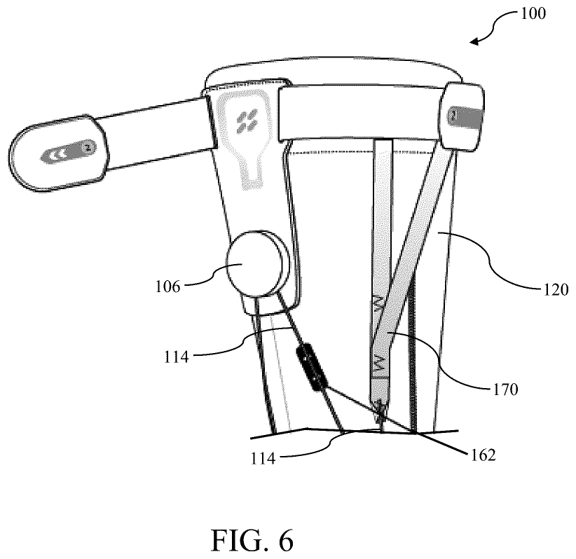

Referring now to FIG. 6, yet another alternative system is illustrated for coupling the top of the sock 120 with the cables (or straps) 114. Specifically, Y-straps 170 interface with the cable (or strap) 114 at one end and are attached to the top of the sock 120 at the other end. While one Y-strap 170 is illustrated in FIG. 6, it would be readily appreciated that a second Y-strap 170 would be integrated on the non-visible side of the sock 120 in some implementations. The Y-strap 170 preferably is coupled to posterior portions of the sock 120 to facilitate pressure being applied around the circumference of the wearer's calf.

Where certain elements of these implementations can be partially or fully implemented using known components, only those portions of such known components that are necessary for an understanding of the present disclosure are described, and detailed descriptions of other portions of such known components are omitted so as not to obscure the disclosure.

In the present specification, an implementation showing a singular component should not be considered limiting; rather, the disclosure is intended to encompass other implementations including a plurality of the same component, and vice-versa, unless explicitly stated otherwise herein.

Further, the present disclosure encompasses present and future known equivalents to the components referred to herein by way of illustration.

It will be recognized that while certain aspects of the technology are described in terms of a specific sequence of steps of a method, these descriptions are only illustrative of the broader methods of the disclosure and may be modified as required by the particular application. Certain steps may be rendered unnecessary or optional under certain circumstances. Additionally, certain steps or functionality may be added to the disclosed implementations, or the order of performance of two or more steps permuted. All such variations are considered to be encompassed within the disclosure disclosed and claimed herein.

While the above detailed description has shown, described, and pointed out novel features of the disclosure as applied to various implementations, it will be understood that various omissions, substitutions, and changes in the form and details of the device or process illustrated may be made by those skilled in the art without departing from the disclosure. The foregoing description is of the best mode presently contemplated of carrying out the principles of the disclosure. This description is in no way meant to be limiting, but rather should be taken as illustrative of the general principles of the technology. The scope of the disclosure should be determined with reference to the claims.

1. An ankle foot orthosis (AFO), comprising:
a sock that is configured to fit over a calf muscle on a leg of a user; and
a lifting cable system that is at least partially integrated within the sock comprising:
a calf strap that is configured to be positioned above the calf muscle of the user when disposed on the leg of the user;
a tensioning structure disposed on an upper region of the sock, the tensioning structure comprising a rotary tensioning mechanism disposed thereon;
an achilles anchor that is configured to be positioned below the tensioning structure on posterior portion of the sock;
a dorsal anchor that is configured to be positioned anterior to the achilles anchor;
a pair of foot anchors being positioned anterior to the dorsal anchor;
a pair of calf anchors that are positioned on an upper posterior portion of the sock; and
a cable that is attached with the rotary tensioning mechanism, the cable being routed through the achilles anchor, the dorsal anchor, the pair of calf anchors and secured to the pair of calf anchors.

2. The AFO of claim 1, wherein the sock comprises a first outer layer of sock and a second inner layer of sock, wherein the achilles anchor, the dorsal anchor, and the pair of foot anchors are positioned between the first outer layer of sock and the second inner layer of sock.

3. The AFO of claim 2, further comprising a toe box positioned anterior to the dorsal anchor on the sock, the toe box being configured to receive toes of the user while wearing the AFO.

4. The AFO of claim 3, wherein the toe box possesses more elasticity in a circumferential direction around the sock as compared with elasticity in a longitudinal direction, the longitudinal direction generally running from a toe region of the sock toward the dorsal anchor, the circumferential direction being orthogonal with the longitudinal direction.

5. The AFO of claim 4, wherein the pair of foot anchors wrap around a foot region of the sock such that portions of the pair of foot anchors reside on both a top region of the sock and a bottom region of the sock.

6. The AFO of claim 5, wherein the pair of foot anchors are disposed between the first outer layer of sock and the toe box.

7. The AFO of claim 2, wherein the calf strap comprises a first end and a second end, wherein the first end and the second end of the calf strap are each configured to be received within respective slots located on the tensioning structure.

8. The AFO of claim 7, wherein the first end of the calf strap is configured to be attached to a medial side of the sock, and the second end of the calf strap is configured to be attached to a lateral side of the sock.

9. The AFO of claim 8, wherein the first end of the calf strap is configured to be attached to the calf strap on the medial side of the sock and the second end of the calf and the second end of the calf strap is configured to be attached to the calf strap on the lateral side of the sock.

10. The AFO of claim 2, wherein an upper portion of the tensioning structure is attached to the upper region of the sock and a lower portion of the tensioning structure is not secured to the sock.

11. The AFO of claim 10, wherein the rotary tensioning mechanism is secured to the lower portion of the tensioning structure.

12. The AFO of claim 1, wherein each of the pair of calf anchors comprises a width and the cable is routed through the width of each of the pair of calf anchors before being secured to itself.

13. The AFO of claim 12, wherein the achilles anchor comprises four cable entry points, with two of the four entry points being located on a medial side of the sock and the other two of the four entry points being located on a lateral side of the sock.

14. The AFO of claim 13, wherein the dorsal anchor comprises four cable entry points, with two of the four entry points being located on the medial side of the sock and the other two of the four entry points being located on the lateral side of the sock.

15. The AFO of claim 14, wherein each of the pair of foot anchors comprises a single cable entry point.

16. The AFO of claim 15, wherein the cable comprises a first cable that is routed on the lateral side of the sock and a second cable that is routed on the medial side of the sock.

17. The AFO of claim 16, wherein the first cable is routed from the rotary tensioning mechanism to a first cable entry point on the achilles anchor, to a first cable entry point on the dorsal anchor, to the single cable entry point on one of the pair of foot anchors, back towards a second cable entry point on the dorsal anchor to a second cable entry point on the achilles anchor and finally to one of the pair of calf anchors.

18. The AFO of claim 17, wherein the second cable is routed from the rotary tensioning mechanism to a third cable entry point on the achilles anchor, to a third cable entry point on the dorsal anchor, to the single cable entry point on a second of the pair of foot anchors, back towards a fourth cable entry point on the dorsal anchor to a fourth cable entry point on the achilles anchor and finally to a second of the pair of calf anchors.

19. The AFO of claim 2, further comprising pull tabs that are positioned posterior from the pair of foot anchors, the pull tabs being configured to assist with donning and/or doffing of the AFO.

20. The AFO of claim 2, wherein a portion of the tensioning structure is disposed between the first outer layer of sock and the second inner layer of sock and another portion of the tensioning structure is not covered by the first outer layer of sock.
